(19) United States
(12) Patent Application Publication
(10) Pub. No.: US 2009/0022781 A1
(43) Pub. Date: Jan. 22, 2009

(54) DELIVERY MEANS

(76) Inventors: Nicholas John Crowther, West Yorkshire (GB); Donald Eagland, West Yorkshire (GB)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(21) Appl. No.: 12/280,254

(22) PCT Filed: Feb. 21, 2007
(86) PCT No.: PCT/GB2007/000592

§ 371 Date: Aug. 21, 2008

(30) Foreign Application Priority Data
Feb. 22, 2006 (GB) 0603487.0

(51) Int. Cl. A61K 33/38; A61F 13/00; A61P 31/00

(52) U.S. Cl. 424/447; 424/618

(57) ABSTRACT

A delivery means, for example a dressing, for delivering an antibacterial metal, for example silver, comprises the metal in combination with a hydrophilic polymer. The polymer may be cross-linked by a butylidene polymer to define a gel. In the examples, silver nitrate may be reduced to metallic silver, protected using polyvinylalcohol which is cross-linked to define a gel.

This invention relates to a delivery means and particularly, although not exclusively, relates to a delivery means for delivering a deliverable material, for example an active material or a precursor thereof, to a locus especially to a wound bed. Preferred embodiments relate to delivery means in the form of wound care devices for delivery of metallic silver to a wound bed.

It is known to incorporate silver-containing active agents into wound care devices to control microbial growth. A diverse range of active silver-containing agents have been proposed. For example U.S. Pat. No. 3,930,000 discloses use of silver zinc allantoinate cream; JP 05179053 discloses use of a silver sodium hydrogen zirconium phosphate. Such complex salts can be expensive to make and difficult to handle.

It is an object of the present invention to address problems associated with known delivery means.

It is another object of the invention to provide a means for delivering a deliverable material a substantial distance into a wound bed.

According to a first aspect of the invention there is provided a delivery means for delivering a deliverable material, said delivery means comprising a deliverable material and a protection means for protecting the deliverable material.

Preferably, said deliverable material comprises a metal. The metal may be an anti-bacterial metal. The metal may be in any suitable form in the delivery means. It may be present as metal ions. Preferably, it is present in the form of a metallic metal—that is in zero oxidation state. The metal may have a positive zeta potential. The zeta potential may be less than 40 mV, preferably less than 35 mV, more preferably less than 30 mV. Zeta potential may be measured by a laser Doppler technique.

Said deliverable material may comprise a precious metal. Said deliverable material may be selected from silver, gold and platinum. It is preferably silver or gold. Most preferably it is silver.

When the deliverable material comprises a metal, suitably at least 50 wt %, preferably at least 70 wt %, more preferably at least 90 wt %, especially at least 95 wt % of the metal is present in its zero oxidation state. In the most preferred embodiment, about 100 wt % of the metal is present in its zero oxidation state. Thus, when the metal comprises silver as described substantially all of the silver is present in the delivery means in its zero oxidation state.

When the deliverable material comprises a metal, the metal is preferably present as substantially pure metal. Thus, it is preferably not present as an alloy.

Said delivery means could include a plurality of deliverable materials, for example a plurality of metals. Suitably at least 50 wt %, preferably at least 70 wt %, more preferably at least 90 wt %, especially at least 95 wt % of the total amount of metals which are deliverable materials comprises metal in a zero oxidation state.

Suitably at least 50 wt %, preferably at least 75 wt %, more preferably at least 95 wt %, especially substantially 10 wt % of the total amount of metal which is deliverable comprises silver, suitably in its zero oxidation state as described.

Suitably at least 50 wt %, preferably at least 70 wt %, more preferably at least 90 wt %, especially at least 95 wt % or even about 100 wt % of the total amount of deliverable materials in said delivery means comprises a metal, especially silver, suitably in its zero oxidation state as described.

Said delivery means preferably comprises colloidal particles of said deliverable material. The number average particle size of said deliverable material (e.g. a metal such as silver) in the device may be in the range 1 to 100 nm, preferably in the range 1 to 50 nm, measured for example using a laser light scattering technique. For the avoidance of doubt, the particle sizes referred to are of the delivery material per se.

Preferably, less than 5 wt %, more preferably less than 1 wt %, of particles of said deliverable material in said delivery means have a particle size of greater than 200 nm.

When said deliverable material comprises silver, as is most preferred, said silver may be present as metallic silver particles, suitably colloidal particles. The silver particles preferably have a positive zeta potential. This may be advantageous in use in an anti-bacterial application since the positively charged particles may more readily be attracted to negatively charged bacteria. The zeta potential may be at least 1 mV and may be 30 mV or less.

Said protection means may be such that it increases the time the deliverable material is in an active form after it has passed outside the delivery means in use. When the delivery means is used to deliver a metal such as silver to a wound (which is one preferred application described herein), the association of the protection means with the metal may increase the distance the metal may diffuse into the wound before being rendered less effective or inactive, for example due to interaction with ionic components of body fluid, for example sodium chloride which in the case of silver would result in formation of a silver chloride precipitate. Thus, when the deliverable material comprises silver, the protection means may be such that it reduces the rate of conversion of the silver to silver chloride by oxidation and/or reaction of the silver with chloride ions present in the wound bed.

Said protection means may restrict oxidation of the deliverable material.

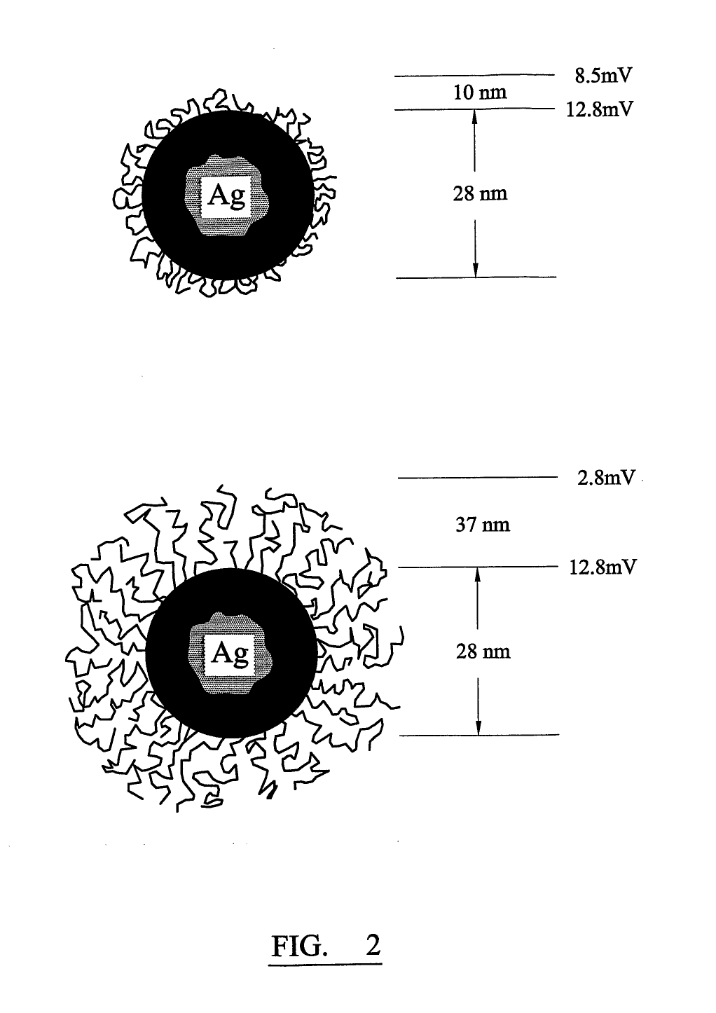

Said protection means preferably comprises a protective layer around particles of deliverable material. The protective layer may be assessed using a laser light scattering technique. It may have a thickness in the range 5 to 100 nm. The thickness may be affected by the strength of interaction between the protection means and the deliverable means. FIG. 2 hereinafter illustrates interaction between a preferred protection means and a preferred deliverable material.

The presence of a protection means may be shown by contacting samples of delivery means which either include or do not include protection means with a reagent which will react with the deliverable material. A sample which includes a protection means may be delayed in reacting with the reagent compared to a sample which is identical except that it does not include protection means. This is illustrated in Example 5 hereinafter.

Said protection means preferably comprises, more preferably consists essentially of, a polymeric material, preferably an organic polymeric material. Preferred polymeric materials comprise atoms selected from carbon, hydrogen, nitrogen and oxygen atoms.

Said protection means may have a maximum solubility in water in the temperature range 0 to 40° C.

Said protection means preferably comprises an optionally derivatised, for example cross-linked, hydrophilic polymer. The hydrophilic polymer may include relatively hydrophilic regions and relatively hydrophobic regions. It is understood that the extent of protection afforded by the protection means to particles of said deliverable material which may pass out of the device, in use, for example into a wound bed, may be related to the relative levels of the hydrophilic and hydrophobic regions in the hydrophilic polymer. In this respect, when the deliverable material comprises metallic metal particles, it is believed to be the hydrophobic regions of the polymer which predominantly bind to the metallic metal particles. The greater the strength of the binding, the greater protection afforded to the particles. Polymers which have relatively large hydrophobic regions may bind more strongly to metallic metal particles compared to polymers with relatively small hydrophobic regions. Also, polymers with a greater % of hydrophobic regions may bind more strongly to metal particles.

Examples of suitable hydrophilic polymers include polymethacrylic acid polymers; polyimides; polyvinylalcohol and copolymers of the aforesaid.

Said hydrophilic polymer preferably includes a carbon atom containing backbone. The carbon atoms are preferably linked together by C—C single bonds. The backbone preferably includes no other types of atoms.

Said hydrophilic polymer preferably includes carbonyl moieties. Such moieties may be included in groups pendent from a backbone of the polymer. Said carbonyl moieties may be components of carboxylic acids or carboxylic acid derivates. Preferably carbonyl moieties are components of ester functional groups, for example groups —OCO—R¹⁰ wherein R¹⁰ represents an optionally-substituted alkyl or alkenyl moiety, especially a C₁₋₄ alkyl or alkenyl moiety. R¹⁰ is preferably an unsubstituted alkyl moiety especially a methyl group. Thus, said hydrophilic polymer preferably includes acetate moieties.

Said hydrophilic polymer preferably includes hydroxyl groups which are suitably pendent from a backbone of the polymer. Preferably hydroxyl groups are bonded directly to the backbone, preferably carbon atoms thereof. Preferred hydroxy groups comprise alcohol functional groups.

Said hydrophilic polymer preferably includes both carbonyl moieties as described and hydroxyl moieties as described, wherein suitably the carbonyl moieties and hydroxyl moieties are present in separate functional groups pendent from the polymer backbone.

Suitably at least 50 mole %, preferably at least 75 mole %, more preferably at least 95 mole %, especially about 100 mole % of said hydrophilic polymer is made up of repeat units which include functional groups which include carbonyl moieties (preferably as part of carboxylic acid or carboxylic acid derivative functional groups) or hydroxyl (especially alcohol) moieties. Suitably, the sum of the mole % of carbonyl containing functional group (e.g. carboxylic acid or carboxylic acid derivative functional groups) and hydroxyl (especially alcohol) functional groups in said hydrophilic polymer is at least 70 mole %, preferably at least 90 mole %, more preferably at least 95 mole %, especially about 100 mole %. Thus, in a preferred embodiment, an hydrophilic polymer material which includes the aforementioned functional groups is not a copolymer which includes other types of functional groups.

Said hydrophilic polymer preferably comprises a polyvinyl polymer. Suitably the sum of the mole % of vinyl moieties in said polymer is at least 70 mole %, preferably at least 90 mole %, more preferably at least 95 mole %, especially about 100 mole %.

The most preferred protection means comprises an optionally-derivatised, for example cross-linked, polyvinylalcohol. Preferred polyvinylalcohols include hydroxyl functional groups which are relatively hydrophilic and acetate functional groups which are relatively hydrophobic. When an optionally-derivatised polyvinylalcohol is used to stabilise a metal such as silver, the acetate groups may predominantly associate with and/or attach to the metal particles to stabilise the particles as described. Polyvinylalcohols which have a relatively low degree of hydrolysation (i.e. have a relatively low level of hydroxyl groups and a high level of acetate groups) may stabilise particles to a greater extent compared to more highly hydrolysed polyvinylalcohols. Thus, silver particles stabilised by optionally-derivatised polyvinylalcohols having a relatively low degree of hydrolysation may be able to diffuse further into a wound bed compared to those stabilised by polyvinylalcohols having a relatively high degree of hydrolysation. The aforementioned is illustrated in the examples hereinafter.

Said protection means preferably comprises an optionally-derivatised polyvinylalcohol which suitably consists essentially of vinylalcohol and vinyl acetate functional groups. Suitably, the polyvinylalcohol is hydrolyzed to an extent of less than 100 mole %, preferably less than 95 mole %. It may be hydrolysed to an extent of at least 10 mole %, preferably at least 25 mole %, more preferably at least 50 mole %, especially at least 60 mole %. Suitably, in said polyvinylalcohol, the ratio of the mole % of vinylalcohol moieties to vinylacetate moieties is at least 0.5, preferably at least 1, more preferably at least 3. The ratio may be less than 10, preferably less than 8.

Preferred polyvinylalcohols have a viscosity (measured on a 4% aqueous solution at 20° C.) of at least 2 mPa.s, preferably at least 4 mPa.s. The viscosity may be less than 100 mPa.s, preferably less than 75 mpa.s,

Said hydrophilic polymer of said protection means is preferably cross-linked by a cross-linking means

A preferred cross-linking means comprises a chemical cross-linking material. Such a material is preferably a polyfunctional compound having at least two functional groups capable of reacting with functional groups of said hydrophilic polymer. Preferably, said cross-linking material includes one or more of carbonyl, carboxyl, hydroxy, epoxy, halogen or amino functional groups which are capable of reacting with groups present along the polymer backbone or in the polymer structure of the hydrophilic polymer. Preferred cross-linking materials include at least two aldehyde groups. Thus, in a preferred embodiment, said protection means includes a material formed by cross-linking polyvinylalcohol using a material having at least two aldehyde groups. Thus, said protection means may include a moiety of formula I.

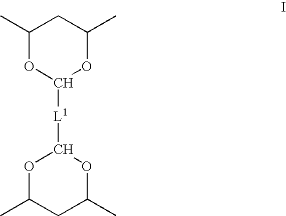

wherein L¹ is a residue of said cross-linking material.

Said cross-linking material preferably comprises a second polymeric material. Said second polymeric material preferably includes a repeat unit of formula

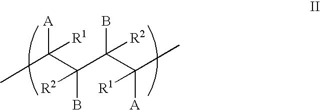

wherein A and B are the same or different, are selected from optionally-substituted aromatic and heteroaromatic groups and at least one comprises a relatively polar atom or group and R¹ and R² independently comprise relatively non-polar atoms or groups.

A and/or B could be multi-cyclic aromatic or heteroaromatic groups. Preferably, A and B are independently selected from optionally-substituted five or more preferably six-membered aromatic and heteroaromatic groups. Preferred heteroatoms of said heteroaromatic groups include nitrogen, oxygen and sulphur atoms of which oxygen and especially nitrogen, are preferred. Preferred heteroaromatic groups include only one heteroatom. Preferably, a or said heteroatom is positioned furthest away from the position of attachment of the heteroaromatic group to the polymer backbone. For example, where the heteroaromatic group comprises a six-membered ring, the heteroatom is preferably provided at the 4-position relative to the position of the bond of the ring with the polymeric backbone.

Preferably, A and B represent different groups. Preferably, one of A or B represents an optionally-substituted aromatic group and the other one represents an optionally-substituted heteroaromatic group. Preferably A represents an optionally-substituted aromatic group and B represents an optionally-substituted heteroaromatic group especially one including a nitrogen heteroatom such as a pyridinyl group.

Unless otherwise stated, optionally-substituted groups described herein, for example groups A and B, may be substituted by halogen atoms, and optionally substituted alkyl, acyl, acetal, hemiacetal, acetalalkyloxy, hemiacetalalkyloxy, nitro, cyano, alkoxy, hydroxy, amino, alkylamino, sulphinyl, alkylsulphinyl, sulphonyl, alkylsulphonyl, sulphonate, amido, alkylamido, alkylcarbonyl, alkoxycarbonyl, halocarbonyl and haloalkyl groups. Preferably, up to 3, more preferably up to 1 optional substituents may be provided on an optionally substituted group.

Unless otherwise stated, an alkyl group may have up to 10, preferably up to 6, more preferably up to 4 carbon atoms, with methyl and ethyl groups being especially preferred.

Preferably, A and B each represent polar atoms or group—that is, there is preferably some charge separation in groups A and B and/or groups A and B do not include carbon and hydrogen atoms only.

Preferably, at least one of A or B includes a functional group which can undergo a condensation reaction, for example on reaction with said hydrophilic polymer. Preferably, A includes a said functional group which can undergo a condensation reaction.

Preferably, one of groups A and B includes an optional substituent which includes a carbonyl or acetal group with a formyl group being especially preferred. The other one of groups A and B may include an optional substituent which is an alkyl group, with an optionally substituted, preferably unsubstituted, C₁₋₄ alkyl group, for example a methyl group, being especially preferred.

Preferably, A represents a group, for example an aromatic group, especially a phenyl group, substituted (preferably at the 4-position relative to polymeric backbone when A represents an optionally-substituted phenyl group) by a formyl group or a group of general formula

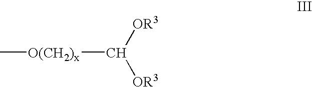

where x is an integer from 1 to 6 and each R³ is independently an alkyl or phenyl group or together form an alkalene group.

Preferably, B represents an optionally-substituted heteroaromatic group, especially a nitrogen-containing heteraromatic group, substituted on the heteroatom with a hydrogen atom or an alkyl or aralkyl group. More preferably, B represents a group of general formula

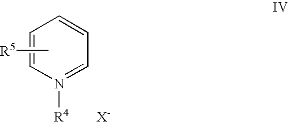

wherein R⁴ represents a hydrogen atom or an alkyl or aralkyl group, R5 represents a hydrogen atom or an alkyl group and X⁻ represents a strongly acidic ion. It is preferably capable of reducing Ag⁺ to Ag⁰ . It may be an organic, for example alkyl, sulphate such a methylsulphate.

Preferably, R¹ and R² are independently selected from a hydrogen atom or an optionally-substituted, preferably unsubstituted, alkyl group. Preferably, R¹ and R² represent the same atom or group. Preferably, R¹ and R² represent a hydrogen atom.

Preferred second polymeric materials may be prepared from any of the following monomers by the method described in WO98/12239 and the content of the aforementioned document is incorporated herein by reference: α-(p-formylstyryl)-pyridinium, γ-(p-formylstyryl)-pyridinium, α-(m-formylstyryl)-pyridinium, N-methyl-α-(p-formylstyryl)-pyridinium, N-methyl-β-(p-formylstyryl)-pyridinium, N-methyl-α-(m-formylstyryl)-pyridinium, N-methyl-α-(o-formylstyryl)-pyridinium, N-ethyl-α-(p-formylstyryl)-pyridinium, N-(2-hydroxyethyl)-α-(p-formylstyryl)-pyridinium, N-(2-hydroxyethyl)-γ-(p-formylstyryl)-pyridinium, N-allyl-α-(p-formylstyryl)-pyridinium, N-methyl-γ-(p-formylstyryl)-pyridinium, N-methyl-γ-(m-formylstyryl)-pyridinium, N-benzyl-α-(p-formylstyryl)-pyridinium, N-benzyl-γ-(p-formylstyryl)-pyridinium and N-carbamoylmethyl-γ-(p-formylstyryl)-pyridinium. These quaternary salts may be used in the form of hydrochlorides, hydrobromides, hydroiodides, perchlorates, tetrafluoroborates, methosulfates, phosphates, sulfates, methane-sulfonates and p-toluene-sulfonates.

Also, the monomer compounds may be styrylpyridinium salts possessing an acetal group, including the following:

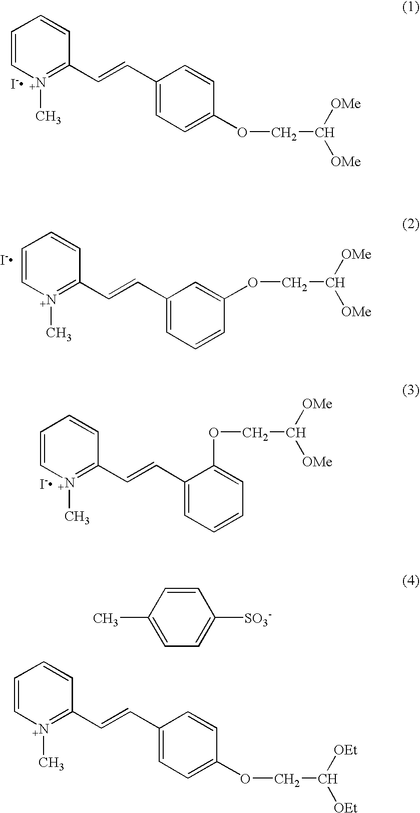

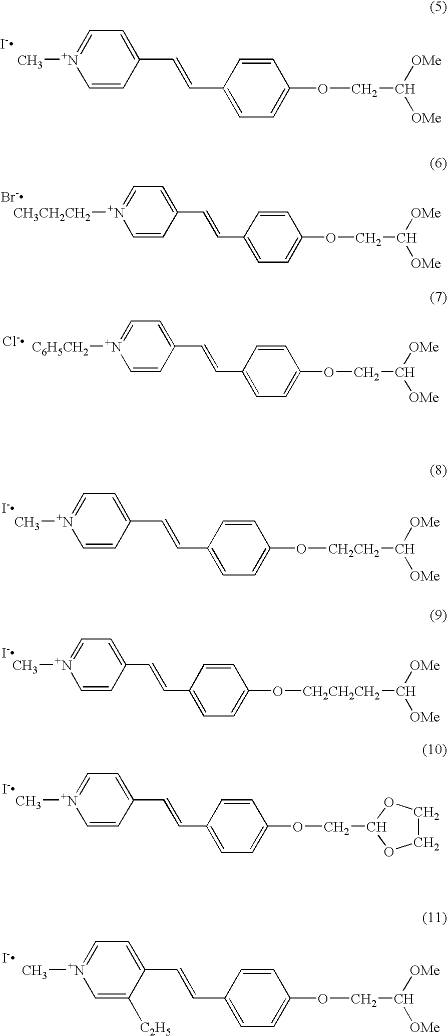

Thus, said second polymeric material is preferably prepared or preparable by providing a compound of general formula

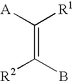

wherein A, B, R¹ and R² are as described above, in an aqueous solvent, (suitably so that molecules of said monomer aggregate) and causing the groups C═C in said compound to react with one another, (for example using UV radiation,) to form said second polymeric material.

Said second polymeric material may be of formula

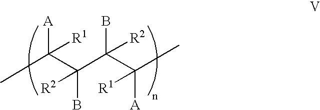

wherein A, B, R¹ and R² are as described above and n is an integer. Integer n is suitably 50 or less, preferably 20 or less, more preferably 10 or less, especially 5 or less. Integer n is suitably at least 1, preferably at least 2, more preferably at least 3.
The ratio of the wt % of said protection means to the wt % of said deliverable material may be at least 10, preferably at least 15, more preferably at least 20. The ratio may be less than 100.

Said protection means and said deliverable material are preferably intimately mixed with one another. Together they preferably define a substantially homogenous mixture.

Said delivery means preferably comprises water.

Said deliverable material is preferably arranged to diffuse within the delivery means. Said deliverable material may be arranged to diffuse out of the delivery means, in use, for example into a wound bed.

Said delivery means preferably comprises a hydrated material. Said delivery means suitably contains at least 2 wt %, preferably at least 25 wt %, more preferably at least 50 wt %, especially at least 80 wt % water. The amount of water may be less than 95 wt %. The level of water may be determined by any suitable means, for example by thermogravimetric analysis.

Said delivery means may include a carrier. Said deliverable material is preferably dispersed within said carrier. Preferably, said carrier and said deliverable material define a substantially homogenous mass comprising deliverable material dispersed within said carrier.

Preferably, said carrier comprises a polymeric material. Such a polymeric material may be naturally-occurring or synthetic. More preferably, it comprise a hydrogel. A said hydrogel may be defined as a cross-linked, water insoluble, water containing material.

Said carrier preferably comprises a polymeric material which is cross-linked by a cross-linking means. Said carrier may be prepared by selecting a first polymeric material and treating it with a said cross-linking means. Said first polymeric material may include functional groups selected from hydroxy, carboxylic acid, carboxylic acid derivatives (e.g. ester) and amine groups. Said first polymeric material preferably includes a backbone comprising, preferably consisting essentially, of carbon atoms. The backbone is preferably saturated. Pendent from the backbone is preferably one or more said functional groups described. Said first polymeric material may have a molecular weight of at least 10,000. Said first polymeric material is preferably a polyvinyl polymer. Preferred first polymeric materials include optionally substituted, preferably unsubstituted, polyvinylalcohol, polyvinylacetate, polyalkylene glycols, for example polypropylene glycol, and collagen (and any component thereof). Polyvinylalcohol is an especially preferred first polymeric material.

Said polyvinylalcohol may be hydrolyzed to an extent of less than 100 mole %, preferably less than 95 mole %. It may be hydrolysed to an extent of at least 10 mole %, preferably at least 25 mole %, more preferably at least 50 mole %, especially at least 60 mole %. Suitably, in said polyvinylalcohol, the ratio of the mole % of vinylalcohol moieties to vinylacetate moieties is at least 0.5, preferably at least 1, more preferably at least 3. The ratio may be less than 10, preferably less than 8.

Said hydrophilic polymer and said first polymeric material preferably comprise the same type of polymeric material. Both preferably comprise a polyvinylalcohol. Preferably, both comprise the same type of polyvinylalcohol.

In especially preferred embodiments said carrier comprises cross-linked polyvinyl alcohol.

Said cross-linking means for cross-linking the polymeric material of said carrier may independently have any feature of the cross-linking means which cross-links said hydrophilic polymer of said protection means. Preferably said cross-linking means of said protection means and of said carrier are substantially the same.

Said delivery means may include less than 20 wt % of said deliverable material. Suitably said delivery means includes less than 10 wt %, preferably less than 5 wt %, more preferably less than 3.5 wt %, especially less than 2 wt % of said deliverable material. Said delivery means may include at least 0.01 ppm, preferably at least 0.1 ppm, more preferably at least 1 ppm of said deliverable material

Said delivery means suitably includes less than 30 wt % of organic polymeric materials (for example said hydrophilic polymer and/or said first and/or second polymeric materials and/or a reaction product thereof), preferably less than 20 wt %, more preferably less than 15 wt %, especially less than 12 wt %. The delivery means may include at least 5 wt %, preferably at least 8 wt % of organic polymeric materials. At least some, suitably at least 50 wt %, preferably at least 75 wt %, more preferably at least 90 wt %, of said organic polymeric material is selected from the group comprising polyvinylalcohol and cross-linked polyvinylalcohol. In said delivery means, the ratio of the sum of wt % of organic polymeric materials to the wt % of said deliverable material is suitably at least 5, and is preferably at least 10. The ratio may be less than 500, preferably less than 250, more preferably less than 100.

Suitably said delivery means comprises:
0.000001 wt % to 5 wt % of a said deliverable material;
5 wt % to 30 wt % of organic polymeric materials; and
65 wt % to 94.999999 wt % of water.
   Suitably, said delivery means comprises:
0.000001 wt % to 5 wt % of a silver (which may be in any form but which preferably comprises a major amount of metallic silver and which most preferably consists essentially of metallic silver);
5 wt % to 30 wt % of polyvinylalcohol and/or cross-linked polyvinylalcohol;
65 wt % to 94.999999 wt % of water.

Preferably, at least some of the polyvinylalcohol of said delivery means is cross-linked.

The delivery means of the first aspect may be in the form of a fluid or in a solid form, for example in the form of a film or sheet.

According to a second aspect of the invention, there is provided a process for preparing a delivery means for delivering a deliverable material, the process comprising the steps of: selecting a deliverable material or a precursor of a deliverable material; and causing said deliverable material or said precursor to be associated with a protection means for protecting the deliverable material.

The process preferably comprises selecting a protection means, for example an optionally cross-linked hydrophilic polymer as described according to said first aspect and contacting the selected material with said deliverable material or precursor. The process preferably comprises intimately mixing the selected protection means and said deliverable material or precursor. Mixing is suitably undertaken in the presence of a liquid, preferably in a liquid which comprises water. Mixing suitably causes the protection means to become associated with said deliverable material or precursor of said deliverable material thereby to stabilise the material. Said protection means may be as described in any statement herein mutatis mutandis. It preferably comprises an optionally cross-linked polyvinylalcohol as described. When said protection means is cross-linked, the process may involve selecting a cross-linking means as described and intimately mixing the selected hydrophilic polymer and selected cross-linking means. Said deliverable material or precursor of said deliverable material may be as described in any statement herein. It may comprise a form of silver or gold. In the event that it comprises metal ions, the process may include a step wherein the metal ions are reduced and in this case the metal ions may be regarded as a precursor of said deliverable material.

The process of the second aspect may comprise forming a carrier in which said deliverable material or precursor of said deliverable material may be dispersed. In this case, the process may comprise causing one or more precursor materials in the presence of a solvent (especially water) to define said carrier.

A first precursor material used in defining said carrier may be a said first polymeric material described according to the first aspect and any feature of said first polymeric material described according to said first aspect may be applied to said second aspect mutatis mutandis. Polyvinylalcohol is an especially preferred first polymeric material as described above.

A second precursor material used in defining said carrier is preferably arranged to cooperate with, preferably to react with, the first precursor material in a step wherein said carrier material is defined. Said second precursor material is preferably a cross-linking means arranged to cross-link the first precursor material. Preferred cross-linking means are chemical cross-linking means as described according to the first aspect. Said second precursor material may comprise a second polymeric material as described according to the first aspect and any feature of said second polymeric material described according to said first aspect may be applied to said second aspect mutatis mutandis.

The process of said second aspect may comprise contacting the first and second polymeric materials in the presence of a solvent, especially water. A catalyst may be present.

Preferably, formation of said carrier from said first and second polymeric materials involves a condensation reaction. Preferably, formation of said carrier involves an acid catalysed reaction. Preferably, said first and second polymeric materials include functional groups which are arranged to react, for example to undergo a condensation reaction, in a step for forming said carrier. Preferably, said first and second polymeric materials include functional groups which are arranged to react, for example to undergo an acid catalysed reaction, in formation of said carrier.

Said deliverable material of the second aspect may be as described according to said first aspect. In one embodiment, fine particles of a metal may be selected and mixed with a selected protection means. Optionally, a said carrier means may then be defined and, in this case, the metal may be substantially chemically unchanged from its selection through to its dispersion in said carrier. In another embodiment, a precursor of said deliverable material may be selected and it may be treated in the method to change its form (e.g. chemical form) so that the deliverable material dispersed in the carrier and the precursor of said deliverable material selected are different. For example, a precursor of said deliverable material may comprise a metal salt and/or a metal in a first oxidation state whereas the deliverable dispersed in the carrier may comprise metallic metal and/or the metal in a different oxidation state.

When a precursor of said deliverable material is selected, the process may utilize means for changing the form (e.g. chemical form) of the precursor of said deliverable material to define the deliverable material in the delivery means. Said means for changing may comprise a chemical means, for example a means to cause a change in oxidation state of said precursor of said deliverable material. Such a means may comprise a reduction means.

The process of the second aspect may be carried out in the presence of a reduction means. Said reduction means may be distinct from means used in said process to define said protection means and, if provided, said carrier. For example, the process may comprise contacting the first and second precursor materials described in the presence of a solvent and in the presence of a reduction means which is different from either said first or second precursor materials. Preferably, however, said reduction means is provided by said first or second precursor materials, especially by said second precursor material. Thus, preferably said second precursor material (especially said second polymeric material described) has multiple roles—cooperation with said first precursor material (e.g. said first polymeric material) to define the carrier, reduction of the precursor of said deliverable material and a cross-linking means of said protection means.

In a preferred embodiment, according to the second aspect, a precursor of said deliverable material is a silver salt, especially silver nitrate, and said salt is reduced in the process, protected by said protection means and dispersed in the carrier. Reduction is preferably caused by a said second precursor material, especially by said second polymeric material referred to herein. In the preferred embodiment, the protection means and the carrier suitably comprise the same type of cross-linked polymeric material.

According to a third aspect of the invention, there is provided a wound dressing comprising a delivery means according to the first aspect or prepared according to the second aspect.

The delivery means of the dressing could be impregnated in a fabric or the like; or the delivery means could be provided in the form of a sheet or film and/or a rigid hydrogel.

The dressing is preferably provided in a substantially sterile package.

According to a fourth aspect, there is provided a method of treating a wound, lesion or other area of a human or animal body which requires treatment, the method comprising contacting an area to be treated with a wound dressing according to the third aspect.

According to a fifth aspect of the present invention, there is provided the use of a delivery means of the first aspect for the manufacture of a dressing for treatment of a wound, lesion or other area of a human body which requires treatment.

Any feature of any aspect of any invention or embodiment described herein may be combined with any feature of any aspect of any other invention or embodiment described herein mutatis mutandis.

Specific embodiments of the invention will now be described, by way of example, with reference to the following figures, in which:

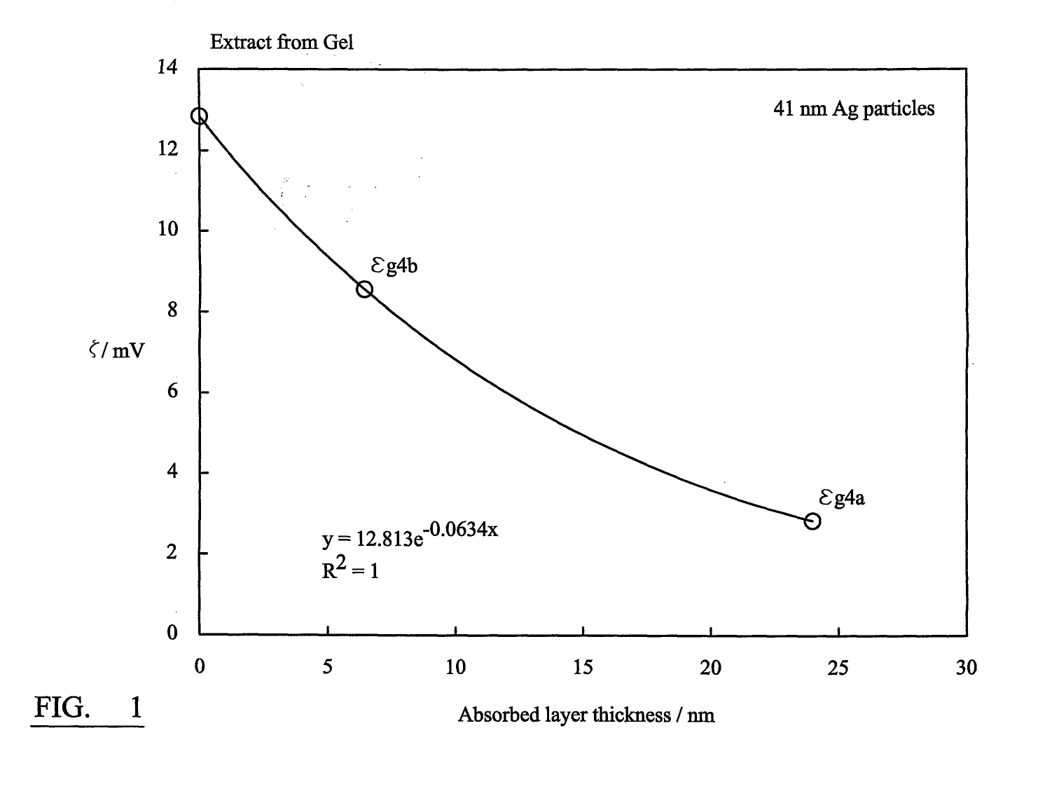

FIG. 1 is a plot of zeta potential vs polyvinylalcohol adsorbed layer thickness for different polyvinylalcohols;

FIG. 2 illustrates KH-20 and Poval 220 molecules binding to Ag⁰ particles;

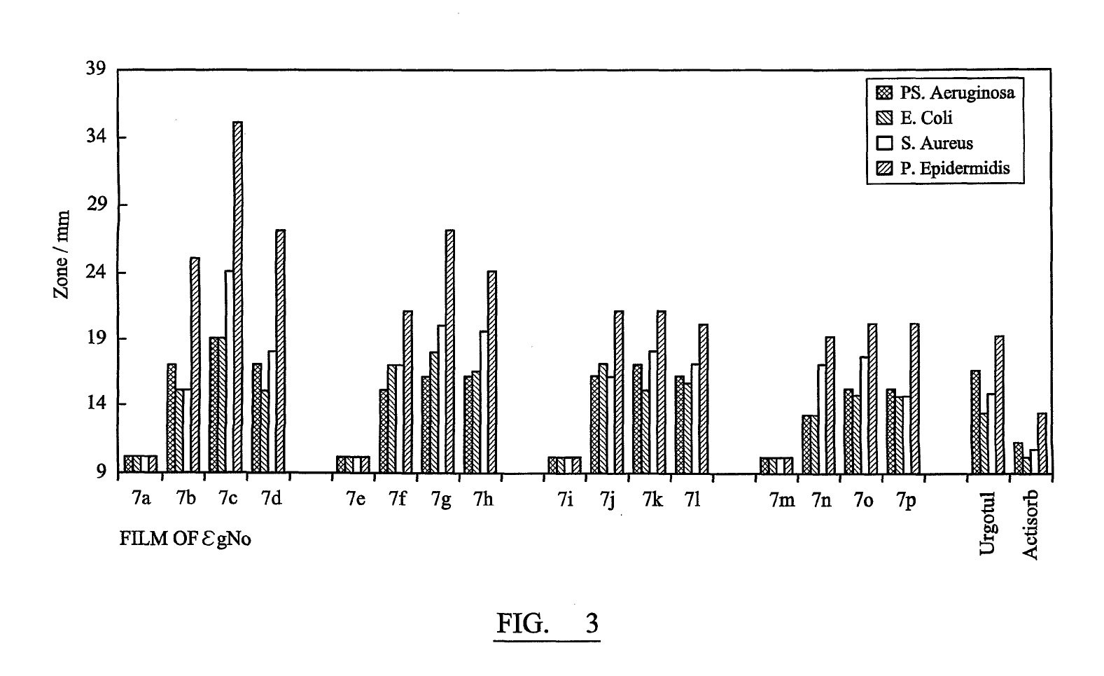

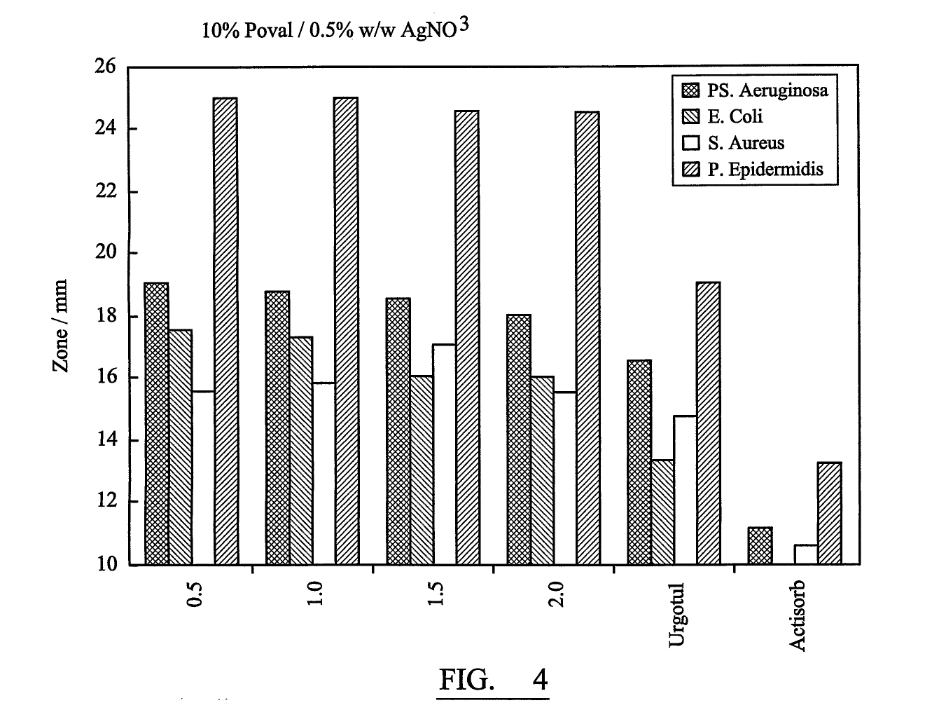

FIGS. 3 and 4 are bar graphs comparing zones of inhibitions of selected materials tested against specified bacteria.

The following materials are referred to hereinafter:

Silver nitrate—refers to an Analar grade;

Poval 220—a polyvinylalcohol obtained from Kuraray having a viscosity, measured on a 4% aqueous solution at 20° C. (determined by a Brookfield synchronised-meter rotary-type viscometer), of 30.mPa.s and a degree of hydrolysis (saponification) of about 88% mol %. The molecular weight is about 130,000.

KP-08 and KH-20 refer to polyvinylalcohols obtained from Marubeni, Speciality Chemicals Inc. KP-08 has a viscosity of 6-8 mPa.s measured as described above and a degree of hydrolysis of 71-73.5 mol %; KH-20 has a viscosity of 44-52 mPa.s and a degree of hydrolysis of 78.5-81.5 mol %.

JF-20—a polyvinyl alcohol obtained from Japan Vam & Poval Co Ltd having a viscosity of 35-45 mPa.s and a degree of hydrolysis of 98.0-99.0 mole %.

Urgotul (Trade Mark) and Actisorb (Trade Mark)—proprietary silver-containing wound dressings.

### EXAMPLE 1

### Preparation of poly (1,4-di(4-(N-methylpyridinyl))-2,3-di(4-(1-formylphenyl)butylidene

This was prepared as described in Example 1 of PCT/GB97/02529, the contents of which are incorporated herein by reference. In the method, an aqueous solution of greater than 1 wt % of 4-(4-formylphenylethenyl)-1-methylpyridinium methosulphonate (SbQ) is prepared by mixing the SbQ with water at ambient temperature. Under such conditions, the SbQ molecules form aggregates. The solution was then exposed to ultraviolet light. This results in a photochemical reaction between the carbon-carbon double bonds of adjacent 4-(4-formylphenylethenyl)-1-methylpyridinium methosulphate molecules (I) in the aggregate, producing a polymer, poly (1,4-di(4-(N-methylpyridinyl))-2,3-di(4-(1-formylphenyl)butylidene methosulphonate (II), as shown in the reaction scheme below. It should be appreciated that the anions of compounds I and II have been omitted in the interests of clarity.

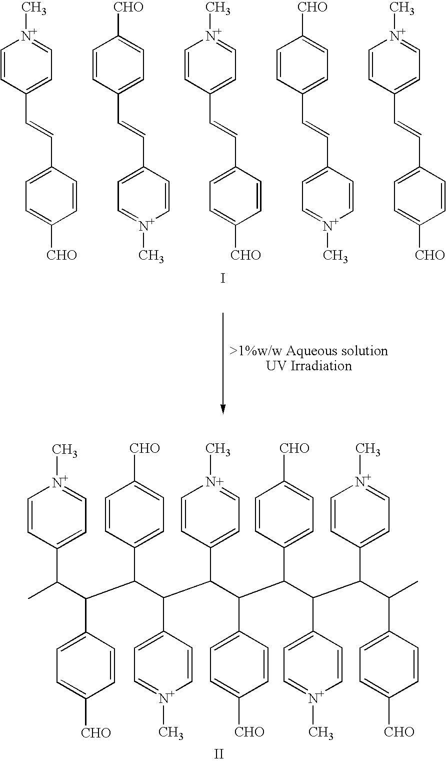

### EXAMPLES 2a-2f

### Preparation of Colloidal Silver

In this example, the preparation of colloidal silver was investigated using the butylidene polymer described in Example 1.

A series of aqueous solutions were prepared having the wt % of silver nitrate and butylidene compound detailed in the table below, the balance being water. Preparation involved addition of aqueous solutions comprising the butylidene polymer to an aqueous solution containing silver nitrate.

| Example No | Wt % of AgNO₃ | Wt % of butylidene |
|---|---|---|
| 2a | 0.5 | 0.125 |
| 2b | 0.5 | 0.25 |
| 2c | 0.5 | 0.5 |
| 2d | 0.5 | 0.75 |
| 2e | 0.5 | 1.0 |
| 2f | 0.5 | 1.5 |

The mixture of Example 2c produced a pale yellow clear solution and there was no sign of precipitation. Dynamic Light Scattering (DLS) showed that the solution contained particles of 54 nm average diameter; the scattering intensity indicated the concentration of the particles was low. The solution was kept in the dark for 24 hours and it was noted that there was a slight increase in particle diameters (to 60 nm) but the concentration of such particles was still low. The solution was then exposed to daylight for 24 hours. DLS then showed that the concentration of particles increased and the particles had an average particle diameter of 41 nm with zeta potential of +12.8 mV

In general terms, solutions of Example 2a to 2f were found to change from pale yellow to darker red brown over a period of 5 hours, when left under normal room lighting. In each case, DLS at 2 to 3 hours after preparation of the solutions showed increasing numbers of particles with diameters 30-40 nm. After 120 hours an equilibrium state was reached.

In conclusion, the investigations undertaken suggest that a photo reduction of all of the Ag⁺ to metallic silver (Ag°) by the methylsulphate anion of the butylidene polymer takes place when silver nitrate and butylidene polymer are contacted in aqueous solution in the light. The silver produced is in the form of positively charged colloidal particles of average particle diameter of the order of 40 nm.

### EXAMPLE 3

### Preparation of Polyvinylalcohol Formulations Containing Silver Nitrate

An aqueous solution comprising 10 wt % Poval 220 polyvinylalcohol and 0.5 wt % of the butylidene polymer of Example 1 was prepared. A typical method for its preparation may comprise dissolving the powderous Poval polyvinylalcohol slowly and with constant stirring in a solution of the butylidene polymer. Complete dissolution may be achieved by maintaining the solution at a temperature of 60° C. for a period of 6 hours. To the solution prepared was added 0.5 wt % of silver nitrate. A clear solution formed which darkens from pale yellow to dark orange over a period of four hours when left in daylight at ambient temperature. There was no visual sign of precipitation.

### EXAMPLES 4a, 4b AND 4c

### Photoreduction of Silver Nitrate

In example 4a, the solution of example 3 was exposed to UV light over a period of 7 to 9 hours. As a result, the Ag⁺ is photoreduced to metallic silver as described in Example 2. In this case, however, polyvinylalcohol is adsorbed onto the silver particles and stabilises them. DLS showed that the solution contained nano particles (of the order of 90 nm diameter) of silver metal diffusing in a viscous polymer solution comprising polyvinylalcohol and butylidene compound. The particles were positively charged and had a zeta potential of +12.8 mV.

In Example 4b, the process of Example 4a was carried out except that, instead of Poval polyvinylalcohol, KH-20 polyvinylalcohol was used.

The materials of Examples 4a and 4b were analysed and a graph of zeta potential against the adsorbed layer thickness of polyvinylalcohol was plotted, as shown in FIG. 1. Referring to the figure, it will be noted that the adsorbed polyvinylalcohol layer thickness for the Poval polyvinylalcohol is significantly greater than for the KH-20 polyvinylalcohol.

In general terms, polyvinylalcohol has large hydrophilic regions and small hydrophobic regions. It is believed that after the reduction of silver ions to metallic silver, the hydrophobic regions of polyvinylalcohol bind to the silver and, accordingly, the polyvinylalcohol stabilises the silver. The Poval polyvinylalcohol is less hydrophobic than the KH-20 (i.e. the Poval has fewer acetate moieties by virtue of it being more highly hydrolysed). As a result, the Poval does not bind as strongly to the silver particles and, therefore, the polyvinylalcohol layer formed using Poval is thicker than that formed using the more strongly binding KH-20.

The binding of the Poval 220 and KH-20 is represented in FIG. 2.

In Example 4c, the procedure of Example 4a was used expect that a 1 wt % solution of Poval was used. Again silver particles were produced in stable colloidal solution. In this case, however, due to the low level of Poval used, the mixture was not as viscous as the other examples

### EXAMPLE 5

### Confirmation of Stabilisation of Silver Particles by Adsorbed Polyvinylalcohol

Sodium chloride solution was added to the photoreduced mixture of example 4a, containing metallic silver nanoparticles. It was found that a white precipitate of silver chloride formed over a period of more than two hours. This shows that the silver nanoparticles are protected by the polyvinylalcohol from immediate reaction with the chloride ions. It has also been observed that colloidal Ag⁰ from sources other than photoreduction as described in Example 4 can be stabilised by polyvinylalcohol in the manner described.

### EXAMPLE 6

### Preparation of Gel

50 g of the formulation of Example 4a, containing silver nano particles, was caused to gel by addition of 0.5 ml of 7% nitric acid. Addition of the acid results in the solution becoming paler. A rigid gel is formed at ambient temperature over a period of about 20-30 minutes. It is believed that gel formation involves cross-linking of polyvinylalcohol chains by the butylidene polymer according to the reaction scheme below.

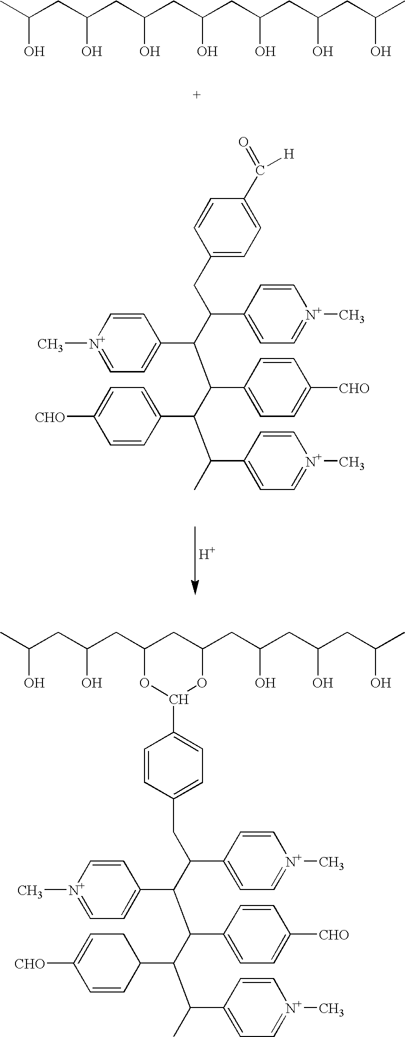

It is believed that the gel prepared comprises silver nano particles which are stabilised by polyvinylalcohol cross-linked by the butylidene polymer. It is believed that the stabilised silver nano particles are freely diffusible within a hydrogel matrix which also comprises polyvinylalcohol cross-linked by the butylidene polymer. This can be illustrated by placing a piece of solid gel in sodium chloride solution. Over a period of time, the sodium chloride solution turns cloudy as silver diffuses from the gel and silver chloride is precipitated.

### EXAMPLE 7

### Preparation of Films for Anti-Bacterial Assessment

A summary of the components used in making films and the concentration of silver nano particles in the films is provided in the table below. In general terms, gels were prepared by mixing 10% w/w of a selected polyvinylalcohol with 0.5% w/w of the butylidene polymer adding a selected amount of silver nitrate, the mixture was allowed to stand for 5 hours in daylight, then acidified with 0.16% nitric acid, poured into 100 mm diameter Petri-dishes to a depth of 3 mm, and allowed to gel for 48 hours. As a result a thin film of gel incorporating Ag⁰ is formed.

| | | Wt % used in preparation | | | |
|---|---|---|---|---|---|
| | Polyvinyl- | (balance water) | | | Wt % of |
| Example | alcohol | Polyvinyl- | Butylidene | Silver | silver in |
| No | type | alcohol | polymer | nitrate | gel |
| 7a | KP-08 | 10 | 0.5 | 0 | 0 |
| 7b | KP-08 | 10 | 0.5 | 0.1 | 0.064 |
| 7c | KP-08 | 10 | 0.5 | 0.5 | 0.32 |
| 7d | KP-08 | 10 | 0.5 | 1 | 0.64 |
| 7e | KH-20 | 10 | 0.5 | 0 | 0 |
| 7f | KH-20 | 10 | 0.5 | 0.1 | 0.064 |
| 7g | KH-20 | 10 | 0.5 | 0.5 | 0.32 |
| 7h | KH-20 | 10 | 0.5 | 1 | 0.64 |
| 7i | Poval | 10 | 0.5 | 0 | 0 |
| 7j | Poval | 10 | 0.5 | 0.1 | 0.064 |
| 7k | Poval | 10 | 0.5 | 0.5 | 0.32 |
| 7l | Poval | 10 | 0.5 | 1 | 0.64 |
| 7m | JF-20 | 10 | 0.5 | 0 | 0 |
| 7n | JF-20 | 10 | 0.5 | 0.1 | 0.064 |
| 7o | JF-20 | 10 | 0.5 | 0.5 | 0.32 |
| 7p | JF-20 | 10 | 0.5 | 1 | 0.64 |

### EXAMPLE 8

### Assessment of Bactericidal Effects of Formulations Comprising Different Amounts of Ag⁰

Petri dishes were filled with nutrient agar, innocculated with bacterial cultures, selected from PS. Aeruginosa, E. Coli, S. Aureus and P. Epidermidis, whilst still molten. The agar was allowed to solidify, for approximately 1 hour, then discs (10 mm diameter) were cut from the silver containing films of Example 7 and placed on the bacteria containing agar, 2 discs per petri dish. This was repeated in triplicate for each bacteria and each film.

The dishes were incubated for 24 hrs at 35° C. Thereafter, the diameters of the zones of inhibition around each film disc were measured and the average taken for each film and each bacteria. For comparative purposes, an analogous procedure was used to measure zones of inhibition for proprietary Urgotul and Actisorb products. Results are recorded in FIG. 3.

It will be noted from FIG. 2 that, in general terms, each of the films of Examples 7b-7d, 7f-7h, 7j-7l and 7m-7p shows a wider zone of inhibition compared to that exhibited by the proprietary Urgotul and Actisorb products. Furthermore, it appears that the lower the degree of hydrolysis (higher acetate content) of the polyvinylalcohols the wider the zone of inhibition which suggests that higher acetate content polyvinylalcohols protect the Ag⁰ better than those of lower acetate content.

### Example 9

### Assessment of Bactericidal Effects of Formulations Comprising Different Amount of Butylidene Polymer

The procedure generally described in Example 8 was followed except that films were prepared using 10 wt % of Poval 220 polyvinylalcohol and 0.5 wt % of silver nitrate and the amount of butylidene polymer was varied from 0.5 wt % to 2.0 wt %. Results are provided in FIG. 4, wherein the wt % of butylidene polymer used to prepare the films is shown on the x axis. The results for proprietary Urgotul and Actisorb products were obtained using 10 mm discs of the commercial dressings placed on the bacteria-containing agar plates.

Referring to FIG. 4, it will be observed that the zone of inhibition is little affected by the level of butylidene polymer used to prepare the films. This may suggest that since the diffusion process is a function of particle size, the butylidene interaction with the polyvinylalcohol has no significant effect upon the thickness of the adsorbed hydrogel layer.

### Example 10

### Preparation of Stabilised Colloidal Gold Formulation

A proprietary aqueous formulation of colloidal gold was selected and mixed with polyvinylalcohol solution so that the concentration of polyvinylalcohol in the aqueous formulation was as much as required to form a solid gel or visco-elastic solution as appropriate. At this stage, the solution was ruby red which is characteristic of colloidal gold. Then, an aqueous solution of the butylidene polymer may be added at a suitable concentration. Where the ratio of the concentration of butylidene polymer to polyvinyl alcohol is in the range 0.1 to 0.05 a visco-elastic solution may be formed after addition of acid. When the concentration of polyvinylalcohol is higher, a solid hydrogel may be formed.

The colloidal gold prepared can be used in a dressing or the like. It is found that, like the silver-containing formulations described, the colloidal gold formulations are protected by the polyvinyl alcohol and/or butylidene polymer allowing them to remain bactericidaly active for longer.

The material described may be incorporated into wound dressings. For example, a fluid may be impregnated in a fabric or the like or a film of the material may be secured to other components of a dressing.

1. A delivery means for delivering a deliverable material, said delivery means comprising a deliverable material and a protection means for protecting the deliverable material.

2. A delivery means according to claim 1, wherein said deliverable material comprises an anti-bacterial metal.

3. A delivery means according to claim 1, wherein said deliverable material comprises silver, gold or platinum.

4. A delivery means according to claim 3, which comprises colloidal particles of said deliverable material.

5. A delivery means according to claim 1, said deliverable material comprising silver, present as colloidal metallic silver particles, wherein the silver particles have a positive zeta potential.

6. A delivery means according to claim 1, wherein said protection means restricts oxidation of the deliverable material and comprises a protective layer around particles of deliverable material.

7. A delivery means according to claim 6, wherein said protection means comprises an organic polymeric material.

8. A delivery means according to claim 3, wherein said protection means comprises an optionally-derivatised hydrophilic polymer.

9. A delivery means according to claim 3, wherein said protection means is selected from optionally-derivatised polymethacrylic acid polymers, polyimides, polyvinylalcohol and copolymers of any of the aforesaid.

10. A delivery means according to claim 3, wherein said protection means comprises an optionally-derivatised polyvinylalcohol.

11. A delivery means according to claim 2, wherein said protection means comprises a polyvinylalcohol which is hydrolysed to an extent of at least 60 mole % and less than 95 mole %.

12. A delivery means according to any preceding claim 1, wherein said protection means comprises a cross-linked water soluble polymer which includes a moiety of formula 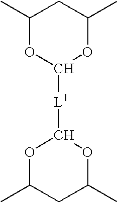
wherein L¹ is a residue of a cross-linking material.

13. A delivery means according to claim 1, wherein said protection means comprises a cross-linked hydrophilic polymer, wherein a cross-linking material used to cross-link the polymer includes a repeat unit of formula 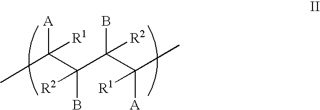
wherein A and B are the same or different, are selected from optionally-substituted aromatic and heteroaromatic groups and at least one comprises a relatively polar atom or group and R¹ and R² independently comprise relatively non-polar atoms or groups.

14. A delivery means according to claim 3, wherein the ratio of the wt % of said protection means to the wt % of said deliverable material is at least 10 and is less than 100.

15. A delivery means according to claim 3, which contains at least 50 wt % water.

16. A delivery means according to claim 15, which includes at least 0.01 wt % and less than 20 wt % of said deliverable material.

17. A delivery means according to any preceding claim 1, which comprises:
0.000001 wt % to 5 wt % of silver in any form;
5 wt % to 30 wt % of polyvinylalcohol and/or cross-linked polyvinylalcohol;
65 wt % to 94.999999 wt % of water.

18. A process for preparing a delivery means as claimed in claim 1, the process comprising the steps of: selecting a deliverable material or a precursor of a deliverable material; and causing said deliverable material or said precursor to be associated with a protection means for protecting the deliverable material.

19. A process according to claim 18, which comprises selecting a protection means and contacting the selected material with said deliverable material or precursor.

20. A process according to claim 19, which comprises selecting a deliverable material in the form of metal ions, wherein said process includes a step wherein the metal ions are reduced.

21. A process according to claim 18, the process comprising forming a carrier in which said deliverable material or a precursor of said deliverable material is dispursed, wherein formation of said carrier involves treating first and second polymeric materials in a condensation reaction.

22. A process according to claim 18, which involves selecting a precursor of a said deliverable material in the form of a silver salt, wherein said salt is reduced in the process, protected by said protection means and dispursed in the carrier.

23. A wound dressing comprising a delivery means according to claim 1 or prepared according to claim 18.

24. A method of treating a wound, lesion or other area of a human or animal body which requires treatment, the method comprising contacting an area to be treated with a wound dressing according to claim 23.

25. (canceled)

26. A delivery means for delivering a deliverable material, said delivery means comprising a deliverable material and a protection means for protecting the deliverable material, wherein said deliverable material comprises silver, gold or platinum and said protection means comprises an optionally derivatised polyvinyl alcohol.

27. A delivery means for delivering a deliverable material, said delivery means comprising a deliverable material and a protection means for protecting the deliverable material, wherein said deliverable material comprises colloidal particles of silver, gold or platinum, wherein said protection means restricts oxidation of the deliverable material and comprises a protective layer around the particles of deliverable material and wherein said protection means comprises an optionally derivatised polyvinyl alcohol.
